(19) United States
(12) Patent Application Publication
(10) Pub. No.: US 2023/0165893 A1
(43) Pub. Date: Jun. 1, 2023

(54) TREATMENT OF VIRAL INFECTIONS

(71) Applicant: SULFAGENIX, INC., Tucson, AZ (US)

(72) Inventors: Gabriel GOJON-ROMANILLOS, San Pedro Garza Garcia (MX); Gabriel GOJON-ZORRILLA, San Pedro Garza Garcia (MX)

(21) Appl. No.: 17/922,477

(22) PCT Filed: May 4, 2021
(86) PCT No.: PCT/US2021/030591

§ 371 Date: Oct. 31, 2022

Related U.S. Application Data
(60) Provisional application No. 63/019,757, filed on May 4, 2020.

(51) Int. Cl. A61K 33/04; A61K 45/06

(52) U.S. Cl. CPC A61K 33/04; A61K 45/06

(57) ABSTRACT

The present invention features treatment of viral infections, e.g., infections caused by enveloped viruses, by administering sulfur-rich compositions.

### BACKGROUND OF THE INVENTION

Viral infections present a tremendous global health threat. When treating a bacterial infection, a number of antibiotics may be used for a single infection, and a single antibiotic may be used to treat many different bacterial infections. In contrast, approved treatments are limited for viral infections. Furthermore, approved antiviral drugs have narrow targets and often effective only against a single viral strain. Another challenge is that viruses rapidly mutate, thereby developing resistance to antiviral therapies. Notwithstanding the specific virus that invades them, mammalian cells all possess the same basic machinery. Thus, an effective antiviral therapeutic requires a dosage that is high enough to damage the virus but not so high that it harms the host. In view of these challenges, an urgent need exists to develop broad spectrum antiviral drugs that have a high barrier to resistance and are effective across a broad range of viral targets.

### SUMMARY OF THE INVENTION

The present invention features compositions and methods for treating viral infections.

In one aspect, the invention features a method of treating or preventing a viral infection in subject caused by an enveloped virus. The method includes administering to the subject a pharmaceutical composition having 90-99.9% (w/w) elemental alpha sulfur, 0.01-10% (w/w) highly polar components, and a pharmaceutically acceptable carrier and/or excipient in an amount and for a duration sufficient to treat or prevent the viral infection. The enveloped virus may be a DNA virus, an RNA virus, or a reverse transcribed virus.

In some embodiments, the enveloped virus is a DNA virus. The DNA virus may be, e.g., a Herpesviridae, Poxviridae, or Pleolipoviridae. The Herpesviridae may be, e.g., Herpes simplex virus, varicella-zoster virus, cytomegalovirus, or Epstein-Barr virus. The Poxviridae may be, e.g., Smallpox virus, cow pox virus, sheep pox virus, orf virus, monkey pox virus, or vaccinia virus.

In some embodiments, the enveloped virus is an RNA virus. The RNA virus may be, e.g., a Togaviridae, Arenaviridae, Flaviviridae, Orthomyxoviridae, Paramyxoviridae, Bunyaviridae, Rhabdoviridae, Filoviridae, Coronaviridae, Bornaviridae, or Arteriviridae. The Togaviridae may be, e.g., rubella virus, eastern equine encephalitis virus, western equine encephalitis virus, Venezuelan equine encephalitis virus, Semliki Forest virus, Sindbis virus, Ross River virus, Barmah Forest virus, Chikungunya virus, Mayaro virus, O'nyong'nyong virus, Unva virus, or Tonate virus. The Arenaviridae may be, e.g., lymphocytic choriomeningitis virus or Lassa fever. The Flaviviridae may be, e.g., Dengue virus, hepatitis C virus, yellow fever virus, West Nile virus, or Zika virus. The Orthomyxoviridae may be, e.g., influenzavirus A, influenzavirus B, influenzavirus C, isavirus, or thogotovirus. The Paramyxoviridae may be, e.g., measles virus, mumps virus, respiratory syncytial virus (RSV), Rinderpest virus, canine distemper virus, or human parainfluenza virus (HPIV). The HPIV may be, e.g., HPIV-1, HPIV-2, HPIV-3, or HPIV-4. The Bunyaviridae may be, e.g., California encephalitis virus or Sin Nombre virus; the Rhabdoviridae is rabies virus or vesicular stomatitis virus. The Filoviridae may be, e.g., Ebola virus or Marburg virus. The Coronaviridae may be, e.g., an alphacoronavirus, betacoronavirus, deltacoronavirus, or gammacoronavirus. The betacoronavirus may be, e.g., Middle East respiratory syndrome-related coronvairus (MERS-CoV), severe acute respiratory syndrome coronavirus (SARS-CoV), or severe acute respiratory syndrome coronavirus 2 (SARS-CoV-2). The Bornaviridae may be, e.g., Borna disease virus. The Arteriviridae may be, e.g., arterivirus or equine arteritis virus.

In some embodiments, the enveloped virus is a reverse transcribing virus. The reverse transcribing virus may be, e.g., a is a Retroviridae or a Hepadnaviridae. The Retroviridae may be, e.g., human immunodeficiency (HIV) virus. The HIV may be HIV-1 or HIV-2. The Hepadnaviridae may be, e.g., Hepatitis B virus.

In some embodiments, the method includes treating a viral infection caused by an enveloped virus that is not HIV or AIDS.

In some embodiments, the method includes treating a viral infection caused by an enveloped virus that is not viral hepatitis.

In some embodiments, the method includes treating a viral infection caused by an enveloped virus that is not RSV.

In some embodiments, the composition includes about 99% (w/w) zerovalent sulfur and about 1% (w/w) highly polar components. The highly polar components may be selected from sodium sulfate, sodium polythionates, and sodium thiosulfate.

In some embodiments, the highly polar components are selected from the group consisting of sodium polythionate, potassium polythionate, ammonium polythionate, calcium polythionate, sodium thiosulfate, potassium thiosulfate, ammonium thiosulfate, calcium thiosulfate, sodium sulfate, potassium sulfate, ammonium sulfate, sodium bisulfite, potassium bisulfite, ammonium bisulfite, calcium bisulfite, sodium chloride, potassium chloride, ammonium chloride, calcium chloride, sodium acetate, sodium palmitate, potassium palmitate, and ammonium laurate.

In some embodiments, the composition includes an elemental alpha sulfur and one or more highly polar components in a ratio from about 10 to about 150 parts elemental alpha sulfur to 1 part highly polar components (w/w) for enteral, topical, or parenteral administration.

In some embodiments, the composition is formulated for enteral administration and the elemental alpha sulfur and the highly polar components are present together in an amount of from about 10 mg to about 10,000 mg, e.g., about 400 mg.

In some embodiments, the composition is a capsule.

In some embodiments, the composition includes a second therapeutic agent. The second therapeutic agent may be an antiviral agent (e.g., an anti-retroviral drug), an antiviral vaccine, an antifungal agent, an antibacterial agent, an anti-inflammatory agent, an antiparasitic agent, a dietary supplement, or a painkiller. The antiviral agent may be, e.g., remdesivir, favipiravir, favilavir, EIDD-2801, galidesivir, SNG001, lopinavir, ritonavir, or a combination thereof. The antibacterial agent may be, e.g., azithromycin or ciproflaxin. The anti-inflammatory agent may be, e.g., tocilizumab. The antiparasitic agent may be, e.g., chloroquine or hydroxychloroquine. The dietary supplement may be, e.g., vitamin C. The painkiller may be, e.g., acetaminophen.

In some embodiments, the composition is produced by providing a first inorganic compound including sulfur in the −2 oxidation state and reacting the first inorganic compound with a second inorganic compound including sulfur in the +4 oxidation state at an acidic pH. The reacting produces a composition that includes (i) 90-99% (w/w) elemental alpha sulfur and 0.01 to 10% (w/w) highly polar components; and (ii) wherein the composition includes at least 96% bioactive zerovalent sulfur that readily undergoes bioconversion into hydrogen sulfide.

### Definitions

By “elemental alpha sulfur” is meant orthorhombic elemental sulfur having the formula S₈. Elemental alpha sulfur exists as S₈ (cyclooctasulfur molecules) but can also include S₇ (cycloheptasulfur molecules) and S₆ (cyclohexasulfur molecules).

By “elemental beta sulfur” is meant monoclinic elemental sulfur having the formula S₈ and consisting mainly of cyclooctasulfur molecules.

By “highly polar component” is meant a compound whose molecules contain at least one ionic bond or one highly polar covalent bond. Highly polar components include, e.g., sodium polythionates, potassium polythionates, ammonium polythionates, calcium polythionates, sodium thiosulfate, potassium thiosulfate, ammonium thiosulfate, calcium thiosulfate, sodium sulfate, potassium sulfate, ammonium sulfate, sodium bisulfite, potassium bisulfite, ammonium bisulfite, calcium bisulfite, sodium chloride, potassium chloride, ammonium chloride, calcium chloride, sodium acetate, sodium palmitate, potassium palmitate, and/or ammonium laurate. Highly polar components also include molecules containing highly polar O—H covalent bonds, e.g., water, alcohols, polyols, polythionic acids, carboxylic acids, and/or sorbitan monostearate. Highly polar components further include compounds whose molecules contain highly polar N—H covalent bonds, for example, primary amines, amino acids, primary amides, peptides and proteins.

By “polythionate” is meant an anion or group of the formula ⁻O₃S—Sₙ—SO₃⁻ (e.g., where n is an integer from 1 to 60, preferably from 1-20, and more preferably 1, 2, 3, 4, 5, or 6).

By “zerovalent sulfur” is meant a sulfur atom with an oxidation state of zero, as calculated according to an agreed-upon set of rules well known to a person skilled in the art (e.g., each cyclooctasulfur molecule (S₈) contains eight zerovalent sulfur atoms, each thiosulfate ion (S₂O₃⁻²) contains one zerovalent sulfur atom, and each polythionate ion contains “n” zerovalent sulfur atoms. Zerovalent sulfur can be found in sulfane sulfur compounds.

By “zerovalent sulfur content” is meant the amount of zerovalent sulfur present in a composition containing elemental alpha sulfur and highly polar components, such as, sodium polythionates, potassium polythionates, ammonium polythionates, calcium polythionates, sodium thiosulfate, potassium thiosulfate, ammonium thiosulfate, calcium thiosulfate, sodium sulfate, potassium sulfate, and ammonium sulfate.

By “sulfane sulfur” is meant a labile, highly reactive sulfur atom at a reduced oxidation state with a valence of 0 or −1, covalently bound to another sulfur atom. Sulfane sulfur compounds can include, e.g., persulfides, polysulfides, polythionates, polysulfanes, thiotaurine, thiosulfate, and/or elemental sulfur. Sulfane sulfur compounds can be formed in the anaerobic cysteine sulfur metabolism with the participation of enzymes such as cystathionase, 3-mercaptopyruvate sulfurtransferase, and/or rhodanese. The last step in enzymatic H₂S-generating pathways generally involves sulfane sulfur-containing species. Compounds containing sulfane sulfur can participate in cell regulation processes through activation or inactivation of enzymes such as, e.g., oxidoreductase containing an iron or molybdenum atom, e.g., xanthine oxidase, aldehyde oxidase, and malate dehydrogenase).

By “enteral” is meant administration that involves any part of the gastrointestinal tract. Enteral administration can include, e.g., by mouth in the form of tablets, capsules, or drops, by gastric feeding tube, duodenal feeding tube, or rectally.

By “topical” is meant administration that is local or systemic, particularly epicutaneous, inhalational, eye drops, and/or ear drops.

By “parenteral” is meant administering the composition of the invention by means other than oral intake, particularly by injection of a form of liquid into the body. Parenteral administration can include, e.g., intravenous, intra-arterial, intraosseous infusion, intra-muscular, intracerebral, intracerebroventricular, and subcutaneous administration.

By “anti-inflammatory drug” is meant an agent or substance that act by reducing inflammation.

By “dietary supplement” is meant an agent, substance, and/or mixture of substances that is a food supplement or nutritional supplement intended to supplement the diet and provide nutrients, such as vitamins, minerals, fiber, fatty acids, or amino acids that may be missing or may not be consumed in sufficient quantities in a person's diet.

By “inorganic” is meant a compound that is not an organic compound.

By “oxidation state” is meant a measure of the degree of oxidation of an atom in a molecule of a substance defined as the charge an atom might be imagined to have when electrons are counted according to an agreed-upon set of rules well known to a person skilled in the art.

By “acid” is meant an Arrhenius acid, a Bronsted-Lowry acid, and/or a Lewis acid. An Arrhenius acid is a substance that increases the concentration of the hydronium ion when dissolved in water. A Bronsted-Lowry acid is a species that donates a proton to a Bronsted-Lowry base. A Lewis acid is a species that accepts a pair of electrons from another species.

By “hydrogen sulfide” is meant a compound having the formula H₂S that is produced in small amounts by many cells of the mammalian body and has a number of biological signaling functions (e.g., a relaxant of smooth muscle, a vasodilator, increases response of NMDA receptor, facilitates long term potentiation, and involvement in memory formation).

By “increasing hydrogen sulfide levels” is meant increasing in the level of hydrogen sulfide produced in the mammalian body (e.g., from cysteine by the enzymes cystathionine beta-synthase and cystathionine gamma-lyase) by at least about 5%, 10%, 20%, 30%, 40%, 50%, 60%, 70%, 80%, 90%, or 100% as compared to a control reference sample. Levels of hydrogen sulfide can be determined using any useful methods known in the art.

By “treating” is meant subjecting a patient to a management regimen for the purpose of combating a disease or disorder and obtaining beneficial or desired results, such as clinical results. Beneficial or desired results can include, but are not limited to, improvement in quality of life, alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable.

By “subject” is meant a mammal (e.g., a human or a non-human).

By “effective amount” of an agent is meant the amount of the agent sufficient to effect beneficial or desired result (e.g., treatment of viral infection), and, as such, an amount of the composition sufficient to achieve an increase in in vivo hydrogen sulfide and/or sulfane sulfur levels, as compared to the level of hydrogen sulfide and/or sulfane sulfur without administration of the composition.

By “composition” is meant a system comprising a substance described herein, optionally formulated with an acceptable excipient, and manufactured or sold with the approval of a governmental regulatory agency as part of a therapeutic regimen for the treatment of disease in a mammal or to promote and maintain general health. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution or colloidal dispersion free of particulate emboli and in a solvent system suitable for intravenous use); or in any other formulation described herein.

By “acceptable excipient” or “acceptable carrier” is meant any ingredient other than the substance described herein (for example, a vehicle capable of suspending or dissolving the active substance and/or substances, e.g., petroleum jelly and polyethylene glycol) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, colloid stabilizers, sweeteners, and water. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, ethylcellulose, gelatin, hydroxypropyl cellulose, hydroxypropyl methylcellulose, lactose, magnesium stearate, maltitol, mannitol, methylcellulose, methyl paraben, microcrystalline cellulose, polyethylene glycol, polyvinyl pyrrolidone, povidone, pregelatinized starch, propyl paraben, shellac, silicon dioxide, sodium carboxymethyl cellulose, sodium starch glycolate, sorbitol, starch (corn), stearic acid, sucrose, talc, titanium dioxide, and xylitol. Excipients may also include diluents (e.g., saline and aqueous buffer solutions), aqueous carriers, and nonaqueous carriers, for example, water, ethanol, polyols (such as glycerol, propylene glycol, polyethylene glycol, and the like), and suitable mixtures thereof, vegetable oils, such as olive oil, and injectable organic esters, such as ethyl oleate.

As used herein, the term “about” means±10% of the recited value.

“Coronavirus” is a virus that infects humans and causes respiratory infection. Coronaviruses can cause pneumonia in subjects and include, without limitation, the betacoronavirus that causes Middle East Respiratory Syndrome (MERS) (also known as MERS-CoV), the betacoronavirus that causes severe acute respiratory syndrome (SARS) (also known as SARS-CoV), and the SARS-CoV-2 virus that causes COVID-19, a respiratory disease characterized by fever, cough, and shortness of breath that may progress to pneumonia and respiratory failure.

As used herein, the term “enveloped virus” refers to a virus that has a lipid bilayer envelope.

As used herein, the term “DNA virus” refers to a virus that has DNA as its genetic material and replicates using a DNA-dependent DNA polymerase.

As used herein, the term “RNA virus” refers to a virus that has RNA as its genetic material and replicates using an RNA-dependent RNA polymerase.

As used herein, the term “reverse transcribing virus” refers to a virus that has RNA as its genetic material and replicates using a reverse transcriptase.

### DETAILED DESCRIPTION

The present invention features compositions and methods for the treatment of viral infections that are caused by enveloped viruses. The compositions described herein include a hydrogen sulfide prodrug of high bioavailability that is particularly useful against enveloped viruses. The highly bioavailable zerovalent-sulfur-rich compositions of the invention contain at least 96% bioactive zerovalent sulfur that readily undergoes bioconversion into hydrogen sulfide. Enveloped viruses are encapsulated by a lipid bilayer, which is derived from the outer membrane of the infected cell. In general, it is easier for a virus to develop resistant to virus-targeted drugs that to host-cell targeted drugs. Hydrogen sulfide and/or hydrogen sulfide derived sulfane sulfur targets the viral envelope. Consequently, it may be difficult for the enveloped virus to develop resistance to the compositions described herein, e.g., via mutation. Furthermore, the compositions may protect cells from oxidative stress induced by a viral infection and boost the immune system of a subject, e.g., having or at risk of having a viral infection. The compositions and methods are described in more detail below.

### Sulfur-Rich Compositions

### Preparations of Highly Bioavailable Zerovalent Sulfur-Rich Compositions

In one embodiment, the highly bioavailable zerovalent-sulfur-rich compositions are obtained by Procedure I outlined below to prepare a 2.7 kg lot of a composition comprising highly bioavailable zerovalent sulfur. The starting materials are listed in Table 1 and suitable equipment is listed in Table 2.

TABLE 1
Starting Materials

| | % | | |
|---|---|---|---|
| | Purity | Weight | Volume |
| Material | (w/w) | (kg) | (L) |
| Anhydrous sodium metabisulfite (Na₂S₂O₅) | 99.4 | 4.890 | |
| Sodium hydrogen sulfide, hydrated* | 70 | 7.090 | |
| (Sodium sulfhydrate, hydrated) | | | |
| Concentrated sulfuric acid | 98 | 6.408 | 3.483 |
| Distilled water | 100 | 67.25 | 67.25 |
| High-purity ice | 100 | 30.0 | |
| Anhydrous ethyl alcohol (free from | ≥99.5 | 5.68 | 7.2 |
| denaturing additives) | | | |

*contains approximately 30% water and 70% NaHS

TABLE 2
Equipment

| Equipment | Preferred specifications |
|---|---|
| 200 L main reaction vessel | plastic or glass-lined |
| 80 L auxiliary reaction vessel | plastic or glass-lined |
| 40 L vessel | plastic or stainless steel |
| 19 L vessel | plastic or stainless steel |
| 10 L vessel | Plastic or stainless steel |
| Large trays | Stainless steel or glass |
| High-torque motor-stirrer | Should be capable of continuously |
| assembly (220 v, 3 HP) with | varying speed between 0 and 1725 rpm. |
| a frequency converter-speed | The speed scale shown in the display |
| control (ABB, model | should go from 0 to 50. The dimensions |
| ACS 150) | of the 304 stainless steel propeller-type |
| | stirrer shaft should be 80 cm long |
| | and 1 inch in diameter. |
| Low-torque stirrer | — |
| pH meter or pH measuring sticks | — |
| Thermometer (−5-110° C.) | — |
| Measuring cylinders | — |
| Scales | — |
| 4 L-Kitasato flask | — |
| Buchner funnel | 185 mm internal diameter |
| Two additional funnels | Graduated |
| At least 2 full-face safety masks | — |
| fitted with cartridges designed to | |
| absorb acid fumes | |
| Vacuum pump or water ejection | — |
| vacuum system | |

### Procedure I

Add portionwise and under brisk stirring, 4.890 kg of Na₂S₂O₅ to 20 L of distilled water contained in the 200 L-main reaction vessel fitted with the high-torque stirrer (7-8 shown in display). The addition is desirably made over 3-5 minutes and an effort should be made to keep the powder from forming lumps. Dissolve 7.090 kg of NaHS.xH₂O in 15 L of distilled water contained in the 80 L auxiliary reaction vessel fitted with the low-torque stirrer. Filter the NaHS solution through 3 pieces of Whatman #1 filter paper under reduced pressure using a Kitasato-Buchner assembly. Collect the filtrates in a 19 L vessel. It should be noted that only a very small amount of impurities is usually retained on the filter papers.

Next, rinse the 80 L auxiliary reaction vessel and transfer the filtered NaHS solution from the 19 L vessel to the 80 L auxiliary reaction vessel. Add 30 L of distilled water to the 80 L auxiliary reaction vessel that contains the filtered NaHS solution. Pour 1.458 kg of concentrated sulfuric acid (98%) into a stirred mixture of 2.25 kg ice and 2.25 kg distilled water contained in a 10 L vessel. The next two steps should take place simultaneously and should last 40 minutes. Pour, at once, 600 ml of Na₂S₂O₅ solution into the auxiliary reaction vessel, which contains the stirred NaHS solution, and start adding (from an addition funnel) the dilute sulfuric acid solution (5.958 kg) into the same vessel with good stirring. Stirring should create a vortex that goes all the way down to the propeller. Wearing a full-face mask (fitted with an acid-absorbing cartridge), add 2.5 kg of ice to the main reaction vessel containing the Na₂S₂O₅ solution. Start pouring concentrated sulfuric acid (4.95 kg) in small portions and under brisk stirring. Alternate acid additions with ice additions so as to prevent the solution from heating up. Measure the temperature of the solutions in both reaction vessels. The temperature of the solution in the 200 L main reaction vessel (Na₂S₂O₅ plus H₂SO₄) should be about 0° C. and the solution in the 80 L auxiliary reaction vessel (NaHS plus a bit of Na₂S₂O₅ plus H₂SO₄) should be between 30-35° C. Charge 5 kg ice into the 200 L reaction vessel (Na₂S₂O₅ plus H₂SO₄) and then run into it the solution contained in the 80 L auxiliary reaction vessel (NaHS plus a bit of Na₂S₂O₅ plus H₂SO₄) under brisk stirring (24.5-25 on speed scale shown in display). This operation should take about 10 minutes and stirring should create a vortex that goes all the way down to the propeller. Upon mixing the 2 solutions, the reaction mixture should go from colorless to canary yellow, fluidity increases, there is some frothing, and a yellowish precipitate separates. Measure the final temperature of the reaction mixture as well as its pH. The temperature should be between 25-30° C. and the pH should be close to 3. Continue stirring briskly for 90 minutes. Stirring should create a vortex that goes all the way down to the propeller.

Allow the reaction mixture to stand undisturbed during 24 hours at room temperature. At the end of this stage the yellowish precipitate should lie at the bottom of the vessel in the form of a relatively compact mass. Without perturbing the precipitate, transfer as much as possible of the liquid phase to a different vessel by decantation or siphoning. Transfer the material remaining in the reaction vessel (about 20 L) to a 40 L plastic or glass container and stir for 1 hour to obtain a homogeneous slurry. Filter the slurry through a #1 Whatman filter paper using a Buchner-Kitasato assembly. Wash the filter cake with 1 L of distilled water or until the filtrate shows no acidity. Washing should be done before the filter cake develops cracks in order to prevent channeling. Immediately after washing keep applying vacuum for 10 more minutes. Over-drying will lead to a highly compact filter cake and will bring about great difficulties in subsequent steps. Use of a rubber or plastic filter dam (or similar device) is recommended. Transfer the relatively dry filter cake to a 10 L plastic container and add 7 L of pure anhydrous ethanol. Stir until all the solid is suspended and keep stirring 1 hour. If the suspension is too thick add more anhydrous ethanol. Filter the suspension through a #1 Whatman filter paper, wash the filter cake with 200 ml of anhydrous ethanol, place the rubber dam on top and keep applying vacuum for no longer than 10 minutes. Over-drying will lead to a highly compact filter cake and will bring about great difficulties in subsequent steps. Transfer the filter cake to large glass or stainless-steel trays for room-temperature air drying. Allow to dry for about 4 days or until constant weight and absence of ethanol odor. The dry product is a material that consists of easily friable lumps and an impalpable powder. Disaggregate the lumps and sift to make sure that the material goes through a 325-standard sieve.

Procedure I yields a product (SG-1002, also referred to as SG1002) containing about 99% zerovalent sulfur and about 1% highly polar components (e.g., sodium sulfate and traces of sodium polythionates and sodium thiosulfate).

In some embodiments, variations of Procedure I may be used to obtain similar materials. Such procedures include but are not limited to the following:

### Procedure II

Use sodium sulfide instead of sodium hydrogen sulfide and adjust the amounts of reactants according to rules well known to those skilled in the art, such as increasing the amount of acid, following the process detailed in Procedure 1.

### Procedure II

Use sodium sulfite instead of sodium metabisulfite and adjust the amount of the reactants according to rules well known to those skilled in the art, following the process detailed in Procedure 1.

### Procedure IV

Use sodium sulfide instead of sodium hydrogen sulfide and sodium sulfite instead of sodium metabisulfite and adjust the amount of the reactants according to rules well known to those skilled in the art, following the process detailed in Procedure 1.

### Procedure V

Use concentrated hydrochloric acid instead of concentrated sulfuric acid with mole-per-mole replacement and following the process detailed in Procedure 1.

### Procedure VI

Use concentrated hydrochloric acid instead of concentrated sulfuric acid with mole-per-mole replacement and following the process detailed in Procedure 11.

### Procedure VII

Use concentrated hydrochloric acid instead of concentrated sulfuric acid with mole-per-mole replacement and following the process detailed in Procedure Ill.

### Procedure VIII

Use concentrated hydrochloric acid instead of concentrated sulfuric acid with mole-per-mole replacement and following the process detailed in Procedure IV.

### Procedure IX

Use potassium salts instead of sodium salts and adjust the amount of the reagents according to rules well known to those skilled in the art, and following the process detailed in Procedure I.

In some embodiments, the reactants used in the procedure can include any compound comprising sulfur in the minus two oxidation state and another compound comprising sulfur in the plus four oxidation state, and optionally an acid and/or catalyst(s).

In other embodiments, vacuum-aided filtration may be replaced by pressure-aided filtration and/or centrifugation. In other embodiments, closed reactors may be used, a heat-exchange cooling system may be substituted for ice addition, spray drying may substitute air drying, and one and/or more steps (e.g., washing with alcohol) may be omitted. It should be understood that embodiments involving a larger or smaller scale of operation are also within the scope of the present invention.

### Characterization of Highly Bioavailable Zerovalent-Sulfur-Rich Compositions

The standard yield of dry, sifted product is 2.7 kg of an impalpable, odorless, fluffy, light yellow, microcrystalline powder with the following properties:
Melting range: the mean melting temperature is between about 117° C. and about 121° C. 2-3° C. (e.g., melting occurs between 118-120° C., 116-119° C., or between 119-120° C.).
Zerovalent sulfur content (w/w): 90-99.9% (e.g., 91%, 92%, 93.5%, 94%, 96%, 96.5%, 97.1%, 97.5%, 98%, 98.6%, 98.9%, or 99.5%)
Elemental alpha sulfur content (w/w): 90-99.9% (e.g., 91%, 92%, 93.5%, 94%, 96%, 97.1%, 97.5%, 98%, 98.6%, 98.9%, or 99.5%)
Highly polar components (w/w): 0.01-10% (e.g., 0.02%, 0.1%, 0.25%, 0.5%, 0.8%, 1%, 1.5%, 2%, 3%, 4%, 5%, 5.5%, 6%, 7%, 8%, 9%, 9.5%, or 9.9%)
Solubility in water at 25° C.: 0%
Solubility in carbon disulfide: 87-97%
Apparent density (tapped): ˜0.6 g/ml
Median particle size distribution: between about 26 and about 33 micrometers (e.g., 26.5, 27, 27.3, 28, 28.5, 29, 29.5, 30, 31.3, 32, 32.5, or 32.9)
Sodium content: ˜0.03%
Oxygen content (by difference): ˜0.12%

The composition obtained by adhering to Procedure I consists of microcrystals rich in zerovalent sulfur; its solubility in carbon disulfide is lower than that of alpha-sulfur (rhombic sulfur) and contains measurable amounts of sodium and oxygen. X-ray diffraction patterns of the composition are consistent with that of alpha sulfur.

The methods used to obtain the data described herein include the following. The solubility of the composition in carbon disulfide was obtained by adding 6 mL of carbon disulfide to 0.500 g of the final product and determining the weight of the residue. Zerovalent sulfur content was measured by sulfitolysis not correcting for the fact that sulfitolysis converts all sulfur atoms in S₈ into thiosulfate but only (n−1) sulfur atoms in Na⁺⁻O₃S—Sₙ— SO₃⁻Na⁺. Sodium content was determined by Galbraith Laboratories, USA (GLI procedure ME-70). Particle size distribution was measured using a Partica LA-950 laser diffraction particle size analyzer from Horiba Instruments.

Without being limited by any hypothesis, it is likely that the high bioavailability of the above material is associated with the hydrophilic nature of the crystal surfaces, which in turn may be related to the presence of highly polar groups such as ^{-SO}₃Na and/or ═SO₃Na₂. These groups might be those present in polythionate molecules (Na⁺⁻O₃S—Sₙ—SO₃⁻Na⁺, e.g., where n=1, 2, or 3), thiosulfates, or sulfates. Highly polar groups such as —SO₃Na may be associated with molecules of water of hydration and may, under some circumstances, undergo cationic exchange, yielding, e.g., ⁻SO₃H groups. Further, the hydrophilicity of the surface of this unique microcrystalline material is in stark contrast with the hydrophobic nature of the surface of crystals of pure alpha- or beta-elemental sulfur. Pure alpha- or beta-elemental sulfur in contrast, is completely soluble in carbon disulfide. Also without being limited by any hypothesis or theory, it is likely that the low bioavailability of ordinary alpha sulfur is directly related to the hydrophobic nature of its surface.

In some embodiments, the composition can be micro- or nanosized, comprising particles rich in alpha sulfur but always modified so as to possess hydrophilic surfaces. Similar compositions also within the scope of the present invention can be obtained by any chemical, electrochemical, mechanochemical, sonochemical, photochemical, microwave-assisted, biochemical and/or biotechnological processes known in the art. Compositions comprising elemental beta sulfur and surface modifying polar groups further constitute embodiments of the present invention. As established, elemental alpha sulfur is converted into beta sulfur when heated and vice versa.

### Determination of Zerovalent Sulfur Content in the Highly Bioavailable Zerovalent Sulfur-Rich Compositions

In one aspect, the zerovalent sulfur content of the composition of the invention can be determined using the method described herein to measure the percentage (w/w) of zerovalent sulfur in alpha sulfur, sodium thiosulfate, and sodium polythionates. The sulfitolysis method for determining zerovalent sulfur content described herein does not correct for the fact that sulfitolysis of polythionate molecules stops at the trithionate as shown in equation (ii), therefore, one of the zerovalent sulfur atoms present in each polythionate molecule escapes sulfitolysis and is not converted into thiosulfate (equation (ii)). However, since the sodium content of the composition disclosed herein is small, the error introduced in the calculation of % zerovalent sulfur is correspondingly small. A detailed analysis of sulfitolysis is described in Koh et al., Anal. Sci. 6:3-14, 1990.

The sulfitolysis reactions equation (i) and (ii) proceed as in the volumetric method for quantitative determination of elemental sulfur in aromatic hydrocarbons reported by Morris et al., Anal. Chem. 20:1037-1039, 1948. The sulfitolysis method described herein is improved compared to the method of Morris et al. in several ways, including the use of n-hexadecyl pyridinium chloride as a sulfitolysis catalyst.

S₈+8SO₃²⁻+8S₂O₃²⁻ Equation (i)

⁻O₃S—Sₙ—SO₃+(n−1)SO₃²⁻→(n−1)S₂O₃²⁻+⁻O₃S—S—SO₃⁻ Equation (ii)

The reagent solutions and methods of preparation of the solutions are shown in Table 3.

TABLE 3
Reagent Solutions and Methods of Preparation

| Reagent Solution | Preparation of Solution |
|---|---|
| Sodium sulfite | Weigh 150 grams of anhydrous chemically |
| (15% w/w) | pure sodium sulfite and dissolved in |
| aqueous solution | 850 mL distilled water. |
| Formaldehyde aqueous | — |
| solution (37%) | |
| 6N HCl | Measure 250 mL of concentrated hydrochloric |
| | acid (approximately 12N) and dilute to |
| | 500 mL with distilled water. |
| Kl (10% w/w in | Weigh 50 grams chemically pure Kl and |
| water) | dissolve in 450 mL distilled water. |
| 0.200N KlO₃ | Weigh 7.134 grams of high purity anhydrous |
| | KlO₃ dissolve in 100 mL distilled |
| | water and dilute to 1 L with distilled water. |
| Hexadecylpyridinium | Weigh 1 gram solid monohydrate and dissolve |
| chloride monohydrate | in 99 mL distilled water. |
| (1% w/w) solution in | |
| water | |
| Soluble starch (5 g/L, | Weigh 1 gram soluble starch, add 10 mg |
| aqueous solution) | red mercuric iodide, add cold water to form a |
| | paste, then add 200 mL boiling water and boil |
| | for 1 or 2 minutes while stirring. Allow the |
| | solution to cool to room temperature. |

To determine the zerovalent sulfur content, weigh 0.160 g±10 mg of the composition into a 250 mL Erlenmeyer flask. Add to the flask 100 mL of 15% Na₂SO₃ solution. Place the flask in a water bath and apply heat until the water boils. Then add 0.5 mL of 1% hexadecylpiridinium chloride monohydrate solution and continue heating until the solid disappears completely. Allow the contents in the flask to cool down to room temperature and place a magnetic stirring bar inside. While stirring, add 15 mL formaldehyde solution, 25 mL 6N solution, 10 mL of 10% KI solution, and 1 mL of 0.5% soluble starch indicating solution. The resulting solution should be colorless. Titrate the contents in the flask with 0.2N KIO₃ solution using a 25 mL burette. As the titration starts, the contents of the flask become amber-colored, but the color disappears quickly. As the equivalence point is approached be very careful not to overstep. The final point is reached when a drop of titrating solution produces no color change.

Titration reaction IO₃⁻+5I⁻+6H⁺+6S₂O₃²⁻+6I⁻+3S₄O₆²⁻+3H₂O Equation (iii):

25% zerovalent sulfur*=(V_{KIO3} (mL)×N_{KIO3}×32.07×100)/(1000×sample weight (g))*susceptible of undergoing sulfitolysis Formula (iv):

### Conditions and Disorders

The compositions described herein can be used to treat a viral infection, e.g., a viral infection caused by an enveloped virus. The enveloped virus may be a DNA virus, an RNA virus, or a reverse transcribed virus.

In some embodiments, the enveloped virus is a DNA virus. The DNA virus may be, e.g., a Herpesviridae, Poxviridae, or Pleolipoviridae. The Herpesviridae may be, e.g., Herpes simplex virus, varicella-zoster virus, cytomegalovirus, or Epstein-Barr virus. The Poxviridae may be, e.g., Smallpox virus, cow pox virus, sheep pox virus, orf virus, monkey pox virus, or vaccinia virus.

In some embodiments, the enveloped virus is an RNA virus. The RNA virus may be, e.g., a Togaviridae, Arenaviridae, Flaviviridae, Orthomyxoviridae, Paramyxoviridae, Bunyaviridae, Rhabdoviridae, Filoviridae, Coronaviridae, Bornaviridae, or Arteriviridae. The Togaviridae may be, e.g., Rubella virus, Eastern equine encephalitis virus, Western equine encephalitis virus, Venezuelan equine encephalitis virus, Semliki Forest virus, Sindbis virus, Ross River virus, Barmah Forest virus, Chikungunya virus, Mayaro virus, O'nyong'nyong virus, Unva virus, or Tonate virus. The Arenaviridae may be, e.g., Lymphocytic choriomeningitis virus or Lassa fever. The Flaviviridae may be, e.g., Dengue virus, hepatitis C virus, yellow fever virus, West Nile virus, or Zika virus. The Orthomyxoviridae may be, e.g., influenzavirus A, influenzavirus B, influenzavirus C, isavirus, or thogotovirus. The Paramyxoviridae may be, e.g., Measles virus, mumps virus, respiratory syncytial virus (RSV), Rinderpest virus, canine distemper virus, or human parainfluenza virus (HPIV). The HPIV may be, e.g., HPIV-1, HPIV-2, HPIV-3, or HPIV-4. The Bunyaviridae may be, e.g., California encephalitis virus or Sin Nombre virus; the Rhabdoviridae is Rabies virus or Vesicular stomatitis virus. The Filoviridae may be, e.g., Ebola virus or Marburg virus. The Coronaviridae may be, e.g., an alphacoronavirus, betacoronavirus, deltacoronavirus, or gammacoronavirus. The betacoronavirus may be, e.g., Middle East respiratory syndrome-related coronvairus (MERS-CoV), severe acute respiratory syndrome coronavirus (SARS-CoV), or Severe acute respiratory syndrome coronavirus 2 (SARS-CoV-2). The Bornaviridae may be, e.g., Borna disease virus. The Arteriviridae may be, e.g., arterivirus or equine arteritis virus.

In some embodiments, the enveloped virus is a reverse transcribing virus. The reverse transcribing virus may be, e.g., a is a Retroviridae or a Hepadnaviridae. The Retroviridae may be, e.g., human immunodeficiency (HIV) virus. The HIV may be HIV-1 or HIV-2. The Hepadnaviridae may be, e.g., Hepatitis B virus.

In some embodiments, the method includes treating a viral infection caused by an enveloped virus that is not HIV or AIDS.

In some embodiments, the method includes treating a viral infection caused by an enveloped virus that is not viral hepatitis.

In some embodiments, the method includes treating a viral infection caused by an enveloped virus that is not RSV.

### Betacoronavirus Infections

The pharmaceutical compositions described herein may be used to treat a betacoronavirus (e.g., MERS-CoV, SARS-CoV, or SARS-CoV-2) infection. Betacoronaviruses are a species of coronavirus that cause respiratory tract infections with extrapulmonary involvement. Betacoronaviruses can be further categorized in four lineages, lineage A (including HCoV-OC43 and HCoV-HKU1), lineage B (including SARS, SARS-CoV-2), lineage C (including BtCoV-HKU4, BtCoV-HKU5, and MERS), and lineage D (including BtCoV-HKU9). These viruses are endemic in human populations and cause more severe disease in neonates, the elderly, and in individuals living with underlying illnesses, with a greater incidence of lower respiratory tract infection in these populations.

SARS-CoV-2 Infection

The pharmaceutical compositions described herein may be used to treat a SARS-CoV-2 infection COVID-19 is a respiratory infection caused by the SARS-CoV-2 coronavirus. SARS-CoV-2 can spread from person to person (e.g., persons who are in close contact with one another (e.g., within six feet)) and through respiratory droplets produced when a person having been infected with the SARS-CoV-2 virus coughs or sneezes and the droplets can come into contact (e.g., contact the nose, the mouth, the eyes, and/or be inhaled into the lungs) with another person thereby exposing the person to the virus. It may also be possible for a person to be exposed to SARS-CoV-2 by touching a surface contaminated with the virus and then touching their own mouth, nose, or their eyes. The incubation period before onset of symptoms of COVID-19 is approximately 2-14 days after exposure to SARS-CoV-2. Symptoms of the disease may include fever, cough, and difficulty breathing. Severity of symptoms may range from mild (e.g., no reported symptoms) to severe illness, including illness resulting in death. The elderly and persons of all ages with underlying health conditions are at higher risk of developing serious illness. A subject may be at risk of having COVID-19 if they have been exposed to someone who has been diagnosed as having the disease, recently travelled to a location experiencing an outbreak of COVID-19, is elderly, or is immunocompromised. A subject can be diagnosed as having COVID-19 by one of skill in the art based on symptoms or a diagnostic test (e.g., an ELISA, lateral flow chromatographic immunoassays to detect SARS-CoV-2 antibodies, or Abbot ID NOW™ platform).

### Pharmaceutical Compositions

The methods described herein include administering a pharmaceutical composition that contains the highly bioavailable zerovalent sulfur-rich compositions or a combination of one of the highly bioavailable zerovalent sulfur-rich compositions described herein and a second therapeutic agent (e.g., anti-inflammatory drug, antibiotic, antiviral (e.g., antiretroviral), and/or a dietary supplement).

A pharmaceutical composition can be administered by a variety of methods known in the art. As will be appreciated by the skilled artisan, the route and/or mode of administration will vary depending upon the desired results. The pharmaceutical compositions can be formulated for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral administration that comprise the above-mentioned agents dissolved, colloidally dispersed, or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like.

The therapeutic composition may be in the form of a solution, colloidal dispersion, a suspension, an emulsion, an infusion device, or a delivery device for implantation or it may be presented as a dry powder to be used as such or to be reconstituted with water or another suitable vehicle before use. The composition can be in the form of a tablet, capsule (e.g., hard gelatin capsule and soft gelatin capsule), liquid, or sustained release tablet for oral administration; or a liquid for intravenous, intrathecal, subcutaneous or parenteral administration; or a cream or ointment for topical administration, or a polymer or other sustained release vehicle for local administration.

Methods well known in the art for making formulations are found, for example, Remington's Pharmaceutical Sciences (2012, 22nd ed.) and in The United States Pharmacopeia: The National Formulary (USP 41 NF 36), published in 2018. Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the substances. Nanoparticulate formulations (e.g., biodegradable nanoparticles, solid lipid nanoparticles, liposomes) may be used to control the biodistribution of the substances. Other potentially useful delivery systems include ethylene-vinyl acetate copolymer particles, osmotic pumps, intrathecal pumps, implantable infusion systems, and liposomes. The concentration of the substance in the formulation varies depending upon a number of factors, including the dosage of the drug to be administered, and the route of administration.

To administer a composition of the invention by certain routes of administration, it may be necessary to coat the composition with, or co-administer the composition with a material to prevent its inactivation. For example, the composition may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7:27-41, 1984). Pharmaceutically acceptable carriers include sterile aqueous solutions or dispersions and sterile powders for the extemporaneous preparation of sterile injectable colloidal solutions or dispersion. The use of such media and agents for pharmaceutically active substances is known in the art and is included in the invention except where any conventional media or agent is incompatible with the active substance. Supplementary active substances can also be incorporated into the compositions.

Therapeutic compositions typically must be sterile and stable under the conditions of manufacture and storage. The composition can be formulated as a suspension, microemulsion, liposome, or other ordered structure suitable to high drug concentration. The carrier can be a solvent or dispersion medium containing, for example, water, ethanol, petroleum jelly (e.g., VASELINE®), polyol (e.g., glycerol, propylene glycol, and liquid polyethylene glycol, and the like), and suitable mixtures thereof, formulated at different percentages (e.g., 5, 10, 15, 20, 25, 30, 35, 40, 45, or 50% by weight in a dispersion medium described herein). The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Colloidal dispersions may be stabilized through addition of agents well known in the art.

The compositions described herein may be sterilized by conventional sterilization techniques or may be sterile filtered. The resulting aqueous dispersions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid or semisolid form may be packaged in multiple single dose units, each containing a fixed amount of the composition, such as in a sealed package of tablets or capsules. The composition in solid form can also be packaged in a container for a flexible quantity, such as in a squeezable tube designed for a topically applicable cream or ointment.

Some formulations of the invention include but are not limited to: preparation of hard gelatin capsules containing, e.g., 100 mg to 400 mg of a highly bioavailable zerovalent-sulfur-rich composition of the invention, preparation of a suspension of about 5-20% (5.5%, 6%, 6.5%, 7%, 8%, 10%, 15%, 17%, or 19%) of highly bioavailable zerovalent-sulfur-rich composition of the invention and petroleum jelly (e.g., Vaseline®) or polyethylene glycol, or a colloidal dispersion of about 5-20% (5.5%, 6%, 6.5%, 7%, 8%, 10%, 15%, 17%, or 19%) of highly bioavailable zerovalent-sulfur-rich composition of the invention in water or oil.

Sterile injectable colloidal suspensions can be prepared by incorporating the active compound in the required amount in an appropriate solvent with one or a combination of ingredients enumerated above, as required, optionally followed by sterilization microfiltration. Generally, dispersions are prepared by incorporating the active compound into a sterile vehicle that contains a basic dispersion medium and the required other ingredients from those enumerated above. Dosage regimens are adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic or prophylactic situation. For example, the compositions of the invention may be administered once or twice weekly by subcutaneous injection or once or twice monthly by subcutaneous injection.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic or prophylactic effect, optionally in association with the required pharmaceutical carrier. The specifications for the dosage unit forms of the invention are dictated by and directly dependent on (a) the unique characteristics of the active substance and the particular therapeutic or prophylactic effect to be achieved, and (b) the limitations inherent in the art of compounding such an active substance for the treatment of sensitivity in individuals.

When the substances of the present invention are administered as pharmaceuticals, to humans and animals, they can be given alone or as a pharmaceutical composition containing, for example, 1 to 100% (more preferably, 10 to 100%, such as 90 to 100%) of active ingredient, optionally in combination with one more pharmaceutically acceptable carriers or excipients.

The compositions containing an effective amount can be administered for prophylactic or therapeutic treatments. In prophylactic applications, compositions can be administered to a patient with a clinically determined predisposition or increased susceptibility to development of a viral infection. Compositions of the invention can be administered to the patient (e.g., a human) in an amount sufficient to delay, reduce, or preferably prevent the onset of the viral infection. In therapeutic applications, compositions are administered to a patient (e.g., a human) already suffering from a viral infection in an amount sufficient to cure or at least partially arrest the symptoms of the viral infection and its complications (e.g., a symptom of the viral infection, e.g., a cough or pneumonia). An amount adequate to accomplish this purpose is defined as a “therapeutically effective dose,” an amount of a compound sufficient to substantially improve some symptom associated with a viral infection. For example, in the treatment of a viral infection, an agent or substance which decreases, prevents, delays, suppresses, or arrests any symptom of the disease or condition would be therapeutically effective. A therapeutically effective amount of an agent or substance is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

When the substances and formulations of this invention are administered in combination therapies with other agents, they may be administered sequentially or concurrently to an individual. Alternatively, pharmaceutical compositions can include a combination of a substance or formulation described herein, optionally in association with a pharmaceutically acceptable excipient, as described herein, and another therapeutic or prophylactic agent known in the art.

The formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### Dosage

The pharmaceutical compositions of the present invention are formulated into pharmaceutically acceptable dosage forms by conventional methods known to those of skill in the art. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of absorption of the particular agent being employed, the duration of the treatment, other drugs, substances, and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian can start doses of the substances of the invention employed in the pharmaceutical composition at levels lower than that required in order to achieve the desired therapeutic effect and gradually increase the dosage until the desired effect is achieved. In general, a suitable daily dose of a composition of the invention will be that amount of the substance which is the lowest dose effective to produce a therapeutic effect. Such an effective dose will generally depend upon the factors described above. The effective daily dose of a therapeutic composition may be administered as two, three, four, five, six or more sub-doses administered separately at appropriate intervals throughout the day, optionally, in unit dosage forms.

Therapeutic dosage levels may include, e.g., from about 10 mg to about 10,000 mg, (e.g., from about 10 mg to about 100 mg, e.g., about 10 mg, 20 mg, 30 mg, 40 mg, 50 mg, 60 mg, 70 mg, 80 mg, 90 mg, or 100 mg, e.g., from about 100 mg to about 1000 mg, e.g., about 200 mg, 300 mg, 400 mg, 500 mg, 600 mg, 700 mg, 800 mg, 900 mg, or 1,000 mg, e.g., from about 1,000 mg to about 2,000 mg, e.g., about 1,100 mg, 1,200 mg, 1,300 mg, 1,400 mg, 1,500 mg, 1,600 mg, 1,700 mg, 1,800 mg, 1,900 mg, or 2,000 mg, e.g., from about 2,000 mg to about 3,000 mg, e.g., about 2,100 mg, 2,200 mg, 2,300 mg, 2,400 mg, 2,500 mg, 2,600 mg, 2,700 mg, 2,800 mg, 2,900 mg, or 3,000 mg, e.g., from about 3,000 mg to about 4,000 mg, e.g., about 3,100 mg, 3,200 mg, 3,300 mg, 3,400 mg, 3,500 mg, 3,600 mg, 3,700 mg, 3,800 mg, 3,900 mg, or 4,000 mg, e.g., from about 4,000 mg to about 5,000 mg, e.g., about 4,100 mg, 4,200 mg, 4,300 mg, 4,400 mg, 4,500 mg, 4,600 mg, 4,700 mg, 4,800 mg, 4,900 mg, or 5,000 mg, e.g., from about 5,000 mg to about 6,000 mg, e.g., about 5,100 mg, 5,200 mg, 5,300 g, 5,400 mg, 5,500 mg, 5,600 mg, 5,700 mg, 5,800 mg, 5,900 mg, or 6,000 mg, e.g., from about 6,000 mg to about 7,000 mg, e.g., about 6,100 mg, 6,200 mg, 6,300 mg, 6,400 mg, 6,500 mg, 6,600 mg, 6,700 mg, 6,800 mg, 6,900 mg, or 7,000 mg, e.g., from about 7,000 mg to about 8,000 mg, e.g., about 7,100 mg, 7,200 mg, 7,300 mg, 7,400 mg, 7,500 mg, 7,600 mg, 7,700 mg, 7,800 mg, 7,900 mg, or 8,000 mg, e.g., from about 8,000 mg to about 9,000 mg, e.g., about 8,100 mg, 8,200 mg, 8,300 mg, 8,400 mg, 8,500 mg, 8,600 mg, 8,700 mg, 8,800 mg, 8,900 mg, or 9,000 mg, e.g., from about 9,000 mg to about 10,000 mg, e.g., about 9,100 mg, 9,200 mg, 9,300 mg, 9,400 mg, 9,500 mg, 9,600 mg, 9,700 mg, 9,800 mg, 9,900 mg, or 10,000 mg) of active zerovalent-sulfur-rich composition. In some embodiments, the therapeutic dosage levels are from about 800 mg to about 1,600 mg (e.g., about 800 mg, 850 mg, 900 mg, 1,000 mg, 1,050 mg, 1,100 mg, 1,200 mg, 1,300 mg, 1,400 mg, 1,450 mg, 1,500 mg, 1,550 mg, or 1,600 mg) of active zerovalent-sulfur-rich composition per day administered orally to adults of average weight afflicted with most of the symptoms, syndromes and pathological conditions described herein, In some embodiments, prophylactic dosage levels are from about 100 mg to about 1,200 mg (e.g., about 100 mg, 110 mg, 140 mg, 200 mg, 250 mg, 300 mg, 350 mg, 400 mg, 460 mg, 700 mg, 750 mg, 800 mg, 900 mg, 1,000 mg, 1,100 mg, 1,150 mg, or 1,200 mg). In some embodiments, the oral dosage levels are about 2,400 mg per day or higher (e.g., about 2,450 mg, 2,500 mg, 3,000 mg, 3,500 mg, 4,000 mg, 8,000 mg, or 10,000 mg) for an adult of average weight. For children, e.g., children with cancer, the dose may be titrated (e.g., the dose may be escalated gradually until signs of gastrointestinal toxicity appear, such as diarrhea or nausea). In preferred embodiments, the highly bioavailable zerovalent-sulfur-rich compositions are extremely safe for oral administration and most patients can tolerate higher dosages as treatment progresses.

In other embodiments, the highly bioavailable zerovalent-sulfur-rich compositions of the invention are safe for topical administration. Acceptable dosage forms for topical administration can be formulated as creams, lotions, pastes, gels, and/or ointments containing the highly bioavailable zerovalent-sulfur-rich compositions.

Final dosage forms suitable for administration to human subjects may comprise one of the highly bioavailable zerovalent-sulfur-rich compositions as pharmacologically-active agent or further comprise other active agents such as alpha-lipoic acid, carnitine, carnitine tartrate, carnitine fumarate, coenzyme Q10, selenium, alpha-ketoglutaric acid, potassium alpha-ketoglutarate, diethyl alpha-ketoglutarate, oxaloacetic acid, sodium oxaloacetate, diethyl oxaloacetate, 2-oxo-3-(ethoxycarbonyl)-pentanodioc acid diethyl ester, L-cystine, paracetamol, a sulfa drug, an NSAID, a corticosteroid, taurine, a vitamin, a prebiotic, another anticancer drug, including but not limited to another mitocan (e.g., a drug targeting the mitochondrial electron transport chain), alkylating agents (e.g. procarbazine, dacarbazine, altretamine, cisplatin), methotrexate, purine antagonists (e.g., mercaptopurine, thioguanine, cladribine, pentostatin), pyrimidine antagonists (e.g., fluorouracil, cytarabine, azacitidine), plant alkaloids (e.g., vinblastine, etoposide, topotecan), hormonal agents (e.g., tamoxifen, flutamide), antibiotics (e.g., doxorubicin, daunorubicin, mitomycin, bleomycin), and mitocans (e.g., sodium dichloroacetate and 3-bromopyruvic acid).

### Combination Therapies

The invention provides methods for treating or preventing a viral infection in a subject by administering to the subject a composition as described herein and one or more additional therapeutic agents. Wherein the subject is treated with a combination therapy of two or more agents, the agents may be administered sequentially (e.g., 1 day apart, 2 days apart, 3 days apart, 1 week apart, 1 month apart, 6 months apart, or more) or substantially simultaneously (e.g., within 1 day). The two or more agents may be formulated a single pharmaceutical composition or may be administered as separate pharmaceutical compositions. The two or more agents may be administered by the same route of administration of different routes of administration. The two or more agents may be administered at the same frequency or different frequencies.

### Antiviral Agents

In some embodiments, the compositions described herein are administered in combination with one or more antiviral agents. In some embodiments, the antiviral agent is remdesivir. In some embodiments, the antiviral agent is favipiravir. In some embodiments, the antiviral agent is favilavir. In some embodiments the antiviral agent is EIDD-2801. In some embodiments, the antiviral is galidesivir. In some embodiments, the antiviral is SNG001. In some embodiments, the antiviral agent is lopinavir, ritonavir, or a combination of lopinavir and ritonavir. In some embodiments, the antiviral is lopinavir. In some embodiments, the antiviral is ritonavir.

### Antiviral Vaccines

In some embodiments, the compositions described herein are administered in combination with a vaccine (e.g., a composition that elicits an immune response in a subject directed against a virus, e.g., a betacoronavirus, such as a SARS-CoV vaccine, a SARC-CoV-2 vaccine, or a MERS-CoV vaccine). The vaccine may be administered substantially simultaneously (e.g., in the same pharmaceutical composition or in separate pharmaceutical compositions) as the composition, or may be administered prior to or following the composition (e.g., within a period of 1 day, 2, days, 5, days, 1 week, 2 weeks, 3 weeks, 1 month, 2 months, 6 months, or 12 months, or more).

### Antifungal Agents

In some embodiments, the compositions described herein are administered in combination with one or more antifungal agents. In some embodiments of the above-described combination therapies for the treatment of infection in a subject in need thereof, the antifungal agent is anidulafungin, caspofungin, micafungin, amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, triazoles, albaconazole, efinaconazole, epoxiconazole, fluconazole, isavuconazole, itraconazole, posaconazole, propiconazole, ravuconazole, terconazole, voriconazole, abafungin, amorolfin, butenafine, naftifine, terbinafine, ciclopirox, flucytosine, griseofulvin, tolnaftate, or undecylenic acid.

### Antibacterial Agents

In some embodiments, any one of the compositions described herein are administered in combination with one or more antibacterial agents. Some embodiments of the above-described combination therapies for the treatment of infection in a subject in need thereof, the antibacterial agent is amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, amoxicillin clavulanate, ampicillin/sulbactam, piperacillin/tazobactam, ticarcillin/clavulanate, bacitracin, colistin, polymyxin b, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (tmp-smx), sulfonamidochrysoidine, demeclocycline, doxycycline, minocycline, oxytetracycline, tetracycline, clofazimine, dapsone, capreomycin, cycloserine, ethambutol(bs), ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim. In some embodiments, the antibacterial agent is azithromycin. In some embodiments, the antibacterial agent is ciproflaxin. The preceding list is meant to be exemplary of antibacterial agents known to one skilled in the art for the treatment of infection and is not meant to limit the scope of the invention.

### Anti-Inflammatory Agents

In some embodiments, the compositions described herein are administered in combination with one or more anti-inflammatory agents. In some embodiments, the anti-inflammatory agent is tocilizumab. In some embodiments, the anti-inflammatory agent is Sarilumab. In some embodiments, the anti-inflammatory is AmnioBoost. In some embodiments, the anti-inflammatory agent is leflunomide. In some embodiments, the anti-inflammatory agent is methotrexate. In some embodiments, the anti-inflammatory agent is sulfasalazine. In some embodiments, the anti-inflammatory agent is abatercept. In some embodiments, the anti-inflammatory agent is rituximab. In some embodiments, the anti-inflammatory agent is tocilizumab. In some embodiments, the anti-inflammatory agent is anakinra. In some embodiments, the anti-inflammatory agent is adalimumab. In some embodiments, the anti-inflammatory agent is etanercept. In some embodiments, the anti-inflammatory agent is infliximab. In some embodiments, the anti-inflammatory agent is certolizumab pegol. In some embodiments, the anti-inflammatory agent is golimumab.

### Antiparasitic Agents

In some embodiments, the antiparasitic agent is hydroxychloroquine. In some embodiments, the antiparasitic agent is chloroquine.

### Dietary Supplements

The composition may be administered in combination with one or more dietary supplements to promote and/or maintain general health. Examples of dietary supplements include, but are not limited to, a vitamin (e.g., Vitamin A, Vitamin B₁, B₂, B₃, B₅, B₆, B₇, B₉, B₁₂, Vitamin C, Vitamin D, Vitamin E, and Vitamin K), a mineral (e.g., potassium, chlorine, sodium, calcium, magnesium, phosphorus, zinc, iron, manganese, copper, iodine, selenium, and molybdenum), an herb or botanical (e.g., St. John's-wort, kava, Shilajit, and Chinese herbal medicines), an amino acid (e.g., glycine, serine, methionine, cysteine, aspartic acid, glutamic acid, glutamine, tryptophan, and phenylalanine), and a concentrate, constituent, extract, and/or a combination of any of the above.

### Painkillers

The composition may be administered in combination with a painkiller or a fever reducer. For example, the composition may be administered with acetaminophen. The composition may be administered with ibuprofen.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

1. A method of treating or preventing a viral infection in subject caused by an enveloped virus, the method comprising administering to the subject a pharmaceutical composition comprising 90-99.9% (w/w) elemental alpha sulfur, 0.01-10% (w/w) highly polar components, and a pharmaceutically acceptable excipient in an amount and for a duration sufficient to treat or prevent the viral infection.

2. The method of claim 1, wherein the enveloped virus is a DNA virus.

3. The method of claim 2, wherein the DNA virus is a Herpesviridae, Poxviridae, or Pleolipoviridae.

4. The method of claim 3, wherein:
the Herpesviridae is Herpes simplex virus, varicella-zoster virus, cytomegalovirus, or Epstein Barr virus; or
the Poxviridae is Smallpox virus, cow pox virus, sheep pox virus, orf virus, monkey pox virus, or vaccinia virus.

5. The method of claim 1, wherein the enveloped virus is an RNA virus.

6. The method of claim 5, wherein the RNA virus is a Togaviridae, Arenaviridae, Flaviviridae, Orthomyxoviridae, Paramyxoviridae, Bunyaviridae, Rhabdoviridae, Filoviridae, Coronaviridae, Bornaviridae, or Arteriviridae.

7. The method of claim 6, wherein:
the Togaviridae is Rubella virus, Eastern equine encephalitis virus, Western equine encephalitis virus, Venezuelan equine encephalitis virus, Semliki Forest virus, Sindbis virus, Ross River virus, Barmah Forest virus, Chikungunya virus, Mayaro virus, O'nyong'nyong virus, Unva virus, or Tonate virus;
the Arenaviridae is Lymphocytic choriomeningitis virus or Lassa fever;
the Flaviviridae is Dengue virus, hepatitis C virus, yellow fever virus, West Nile virus, or Zika virus;
the Orthomyxoviridae is influenzavirus A, influenzavirus B, influenzavirus C, isavirus, or thogotovirus;
the Paramyxoviridae is Measles virus, mumps virus, respiratory syncytial virus (RSV), Rinderpest virus, canine distemper virus, or human parainfluenza virus (HPIV);
the Bunyaviridae is California encephalitis virus, or Sin Nombre virus;
the Rhabdoviridae is Rabies virus or Vesicular stomatitis virus;
the Filoviridae is Ebola virus or Marburg virus;
the Coronaviridae is an alphacoronavirus, betacoronavirus, deltacoronavirus, or gammacoronavirus;
the Bornaviridae is Borna disease virus; or
the Arteriviridae is arterivirus or equine arteritis virus.

8. The method of claim 7, wherein HPIV is HPIV-1, HPIV-2, HPIV-3, or HPIV-4.

9. The method of claim 7, wherein the betacoronavirus is Middle East respiratory syndrome-related coronavirus (MERS-CoV), severe acute respiratory syndrome coronavirus (SARS-CoV), or severe acute respiratory syndrome coronavirus 2 (SARS-CoV-2).

10. The method of claim 9, wherein the betacoronavirus is SARS-CoV-2.

11. The method of claim 1, wherein the enveloped virus is a reverse transcribing virus.

12. The method of claim 11, wherein the reverse transcribing virus is a Retroviridae or a Hepadnaviridae.

13. The method of claim 12, wherein:
the Retroviridae is human immunodeficiency (HIV) virus; or
the Hepadnaviridae is Hepatitis B virus.

14. The method of claim 13, wherein the HIV is HIV-1 or HIV-2.

15. The method of claim 1, wherein the composition comprises about 99% (w/w) zerovalent sulfur and about 1% (w/w) highly polar components, wherein the highly polar components are selected from sodium sulfate, sodium polythionates, and sodium thiosulfate.

16. The method of claim 1, wherein the highly polar components are selected from the group consisting of sodium polythionate, potassium polythionate, ammonium polythionate, calcium polythionate, sodium thiosulfate, potassium thiosulfate, ammonium thiosulfate, calcium thiosulfate, sodium sulfate, potassium sulfate, ammonium sulfate, sodium bisulfite, potassium bisulfite, ammonium bisulfite, calcium bisulfite, sodium chloride, potassium chloride, ammonium chloride, calcium chloride, sodium acetate, sodium palmitate, potassium palmitate, and ammonium laurate.

17. The method of claim 1, wherein the composition comprises an elemental alpha sulfur and one or more highly polar components in a ratio from about 10 to about 150 parts elemental alpha sulfur to 1 part highly polar components (w/w) for enteral, topical, or parenteral administration.

18. The method of claim 17, wherein the composition is formulated for enteral administration and the elemental alpha sulfur and the highly polar components are present together in an amount of about 400 mg.

19. The method of claim 1, wherein the composition is a capsule.

20. The method of claim 1, wherein the composition comprises a second therapeutic agent.

21. The method of claim 20, wherein the second therapeutic agent is an antiviral agent, an antiviral vaccine, an antifungal agent, an antibacterial agent, an anti-inflammatory agent, an antiparasitic agent, a dietary supplement, or a painkiller.

22. The method of claim 21, wherein:
the antiviral agent is remdesivir, favipiravir, favilavir, EIDD-2801, galidesivir, SNG001, lopinavir, ritonavir, or a combination thereof;
the antibacterial agent is azithromycin or ciproflaxin;
the anti-inflammatory agent is tocilizumab;
the antiparasitic agent is chloroquine or hydroxychloroquine;
the dietary supplement is vitamin C; or
the painkiller is acetaminophen.

23. The method of claim 1, wherein the composition is produced by:
(a) providing a first inorganic compound comprising sulfur in the −2 oxidation state; and
(b) reacting the first inorganic compound with a second inorganic compound comprising sulfur in the +4 oxidation state at an acidic pH, wherein the reacting produced a composition comprising:
(i) 90-99% (w/w) elemental alpha sulfur and 0.01 to 10% (w/w) highly polar components; and
(ii) wherein the composition comprises at least 96% bioactive zerovalent sulfur that readily undergoes bioconversion into hydrogen sulfide.
